(19) United States
(12) Patent Application Publication
(10) Pub. No.: US 2011/0287113 A1
(43) Pub. Date: Nov. 24, 2011

(54) TREATMENT OF PSORIASIS

(75) Inventors: Paul Davis, Bedford (GB); Andrew Austin, Wellingborough (GB); Jan Jezek, Stanwick (GB)

(73) Assignee: Insense Limited, Bedford (GB)

(21) Appl. No.: 13/146,725

(22) PCT Filed: Jan. 22, 2010
(86) PCT No.: PCT/GB10/50091

§ 371 Date: Jul. 28, 2011

(30) Foreign Application Priority Data
Jan. 29, 2009 (GB) 0901456.4

(51) Int. Cl. A61K 33/00; A61P 17/06

(52) U.S. Cl. 424/718

(57) ABSTRACT

The invention relates to an aqueous composition capable of delivering nitric oxide for use in the treatment of psoriasis, particularly plaque psoriasis, e.g. by application of a skin dressing.

### TECHNICAL FIELD

The present invention relates to a method of treatment of psoriasis, particularly plaque psoriasis, on the human or animal body.

### BACKGROUND

Psoriasis is an inflammatory skin disease in which skin cells replicate at an extremely rapid rate. New skin cells are produced about eight times faster than normal, over several days instead of a month, but the rate at which old cells slough off is unchanged. This causes cells to build up on the skin's surface, forming thick patches, or plaques, of red sores (lesions) covered with flaky, silvery-white dead skin cells (scales).

Psoriasis appears in a variety of forms, each having distinct characteristics. Typically, people have only one type of psoriasis at a time, but occasionally two or more different types of psoriasis can occur at the same time. Psoriasis can also occasionally change from one form to another.

Plaque psoriasis (psoriasis vulgaris) is the most prevalent form of the disease. About 80 percent of all those who have psoriasis have this form. It is characterised by raised, inflamed, red lesions covered by a silvery white scale. It is typically found on the elbows, knees, scalp and lower back.

Guttate psoriasis is a form of psoriasis that often starts in childhood or young adulthood. This form of psoriasis resembles small, red, individual spots on the skin and usually appears on the trunk and limbs. These spots are not usually as thick as plaque lesions.

Inverse psoriasis is found in the armpits, groin, under the breasts, and in other skin folds around the genitals and the buttocks. This type of psoriasis first shows up as lesions that are very red and usually lack the scale associated with plaque psoriasis. It may appear smooth and shiny.

Pustular psoriasis, primarily seen in adults, is characterized by white pustules (blisters of noninfectious pus) surrounded by red skin. It may be localized to certain areas of the body—for example, the hands and feet. Pustular psoriasis also can be generalized, covering most of the body. It tends to go in a cycle, first reddening of the skin followed by formation of pustules and scaling.

Erythrodermic psoriasis is a particularly inflammatory form of psoriasis that often affects most of the body surface. It may occur in association with pustular psoriasis. It is characterized by periodic, widespread, fiery redness of the skin. The erythema (reddening) and exfoliation (shedding) of the skin are often accompanied by severe itching and pain.

Recent studies have shown that psoriasis can have a substantial impact on quality of life, even in patients with low severity psoriasis.
Psoriasis affects an estimated 2-3 percent of the world's population.
125 million people worldwide have psoriasis, according to the World Psoriasis Day consortium.
According to the US National Institutes of Health (NIH), between 5.8 and 7.5 million Americans have psoriasis, a prevalence of over 2%.
A prevalence of 1.5% has been identified in the UK (Gelfand et al, 2005).

Psoriasis is exceeded only by congestive heart failure in patient-reported physical disability scores on the Short Form (SF-36) Health Survey. This means that psoriatics sufferers experience greater physical disability than people with hypertension, myocardial infarction, diabetes, depression, arthritis, or cancer. Only depression exacts a higher toll than psoriasis, as indicated by scores on the mental health component of the SF-36 (Leonardi C L, 2003).

Although the causes of psoriasis are not fully understood, the evolving evidence suggests that psoriasis is a complex disorder caused by the interaction of multiple genes, the immune system, and environmental factors.

Researchers have found 9 gene mutations that may be involved in causing psoriasis. One of these mutations on chromosome 6, called PSORS-1, appears to be a major factor that can lead to psoriasis. Mutations on genes cause certain cells to function differently. With psoriasis, these mutations seem to largely affect T-helper cells.

Immune system dysfunction in psoriasis is characterized by an abnormal regulation of the interaction between T cells and keratinocytes. One of the central immunological mediators in psoriasis is the cytokine TNF-α. It is one of the major naturally occurring cytokines in the skin, and is involved in several normal and abnormal inflammatory immune responses, and is found in elevated levels in the skin of psoriatic patients. TNF-α directly affects the pathogenesis of psoriasis, and demonstrates this by inducing the synthesis of adhesion molecules on endothelial cells and keratinocytes. This process thereby influences cellular infiltration in the skin, and has a direct effect on the abnormal keratinocyte proliferation and maturation seen in psoriatic lesions.

A variety of topical agents are recommended for mild to moderate psoriasis.

Topical corticosteroids are the most commonly used topicals to treat psoriasis and can be very effective in controlling mild to moderate psoriasis lesions as well as having a rapid onset-of-action. They retard the growth of skin cells and reduce inflammation. Some steroids are potent but also cause skin damage if used too frequently. They are available in a large range of vehicles including powders, sprays, gels, creams, and even foam vehicles for use on the scalp. However, one of the most troubling features of topical corticosteroids is that patients develop tachyphylaxis, a phenomenon whereby medications that are highly effective initially, lose efficacy with prolonged use.

The second most commonly used topicals are vitamin D3 analogues, which slow down the rate of skin cell growth, flatten psoriasis lesions and remove scale. They can also be used on scalp and nail psoriasis. Although efficacy is comparable to that of potent corticosteroids without the attendant risks, onset-of-action is slow and skin irritation common (about 20%-25% of users), hence the utility of combination therapy with corticosteroids that tends to abrogate both these problems (Smith and Barker, 2006).

For moderate and severe psoriasis phototherapy is recommended.

UVB treatment involves exposing the skin to an artificial UVB light source (315-280 nm) for a set length of time on a regular schedule, either under a doctor's direction in a medical setting or with a home unit purchased with a doctor's prescription. Ultraviolet B (UVB) (315-280 nm) is absorbed by the epidermis and has a beneficial effect on psoriasis. Narrowband UVB (311 to 312 nm), is that part of the UVB spectrum that is most helpful for psoriasis

Ultraviolet light treatment is frequently combined with topical (coal tar, vitamin D3 analogues) or systemic treatment (retinoids) as there is a synergy in their combination. The Ingram regime, involves UVB and the application of anthralin paste. The Goeckerman regime combines coal tar ointment with UVB.

Psoralen and ultraviolet A phototherapy (PUVA) combines the oral or topical administration of psoralen with exposure to ultraviolet A (UVA) light. Precisely how PUVA works is not known. The mechanism of action probably involves activation of psoralen by UVA light, which inhibits the abnormally rapid production of the cells in psoriatic skin. There are multiple mechanisms of action associated with PUVA, including effects on the skin immune system.

Excimer laser (known by brand names Xtrac Ultra and Xtrac Velocity), which is approved by the FDA for psoriasis, emits a high-intensity beam of UV light that can be targeted at selected areas of the skin affected by psoriasis. Mostly, the laser is used to treat people with mild to moderate plaque psoriasis.

Like the excimer lasers, pulsed dye lasers are approved for treating chronic, localized plaque lesions. Pulsed dye lasers destroy the tiny blood vessels that contribute to and support the formation of psoriasis lesions. They have been in use for approximately 15 years for removing unwanted blood vessels and birthmarks, such as port wine stains. Investigators first reported that psoriasis could be cleared with pulsed dye lasers in 1990.

Treatment with a pulsed dye laser reportedly feels like being snapped repeatedly with a rubber band. Treatment consists of 15- to 30-minute sessions every three weeks. For patients who respond, usually it takes between four and six sessions to clear the target lesion. Side effects of pulsed dye laser treatments include a small risk of scarring. The most common side effect is a bruise that remains after treatment for a week to 10 days.

For the most severe psoriasis systemic treatment is recommended.

The three main traditional systemic treatments are methotrexate, cyclosporine and retinoids. Methotrexate and cyclosporine are immunosupressant drugs; retinoids are synthetic forms of vitamin A. Other additional drugs, not specifically licensed for psoriasis, have been found to be effective. These include the antimetabolite tioguanine, the cytotoxic agent hydroxyurea, sulfasalazine, the immunosupressants mycophenolate mofetil, azathioprine and oral tacrolimus. These have all been used effectively to treat psoriasis when other treatments have failed. Although not licensed in many other countries fumaric acid esters have also been used to treat severe psoriasis in Germany for over 20 years.

Recently licensed biological treatments, such as efalizumab (anti-adhesion antibody), etanercept (anti-TNF alpha), and infliximab (anti-TNF alpha), provide a major advance in treatment but are currently indicated for limited severe disease owing to lack of data on long term safety and efficacy, and cost.

In addition to the above conventional treatments, various alternative therapies have been suggested. Everything from lifestyle, acupuncture, herbal remedies, meditation and magnets, to the use of doctor fish (which live in the outdoor pools of spas and are encouraged to feed on the psoriatic skin) are implicated in being effective at managing psoriasis.

It can therefore be seen that effective treatments with reduced side effects would be enormously desirable.

WO 2008/048514 discloses the application of gaseous nitric oxide for treatment of a variety of skin conditions, including psoriasis.

### SUMMARY OF THE INVENTION

In a first aspect, the invention relates to a method of treatment of psoriasis on the human or animal body, comprising applying an aqueous composition capable of delivering nitric oxide.

In a second aspect, the invention relates to an aqueous composition capable of delivering nitric oxide for use in the treatment of psoriasis on the human or animal body.

In a third aspect, the invention relates to the use of an aqueous composition capable of delivering nitric oxide in the manufacture of a medicament for the treatment of psoriasis on the human or animal body.

The present inventors have surprisingly discovered that psoriasis symptoms, particularly plague psoriasis, show dramatic improvements when an aqueous composition capable of delivering nitric oxide is applied to it, without any significant side-effects.

Under normal conditions, nitric oxide (NO) is a short-lived, reactive gaseous substance. Its reactivity is due to the unpaired electron of nitrogen. As a molecule with an unpaired electron, nitric oxide can be described as a free radical. However, compared with typical free radicals (e.g. hydroxyl radical or superoxide), whose life-time is in the order of milliseconds, nitric oxide is relatively stable. Typically, it is converted to a more stable chemical species within seconds of its production. Thus, for example, if gaseous nitric oxide contacts air, it reacts rapidly with oxygen to generate nitrogen dioxide as follows:

2NO+O₂→2NO₂ (1)

Under some conditions, for instance in pure gaseous state, NO can be stored without significant losses for a very long time. NO is a very hydrophobic compound and its solubility in water is therefore limited. Maximum solubility in water achievable under normal conditions is approximately 1.7 mM, the solubility being similar to that of oxygen. The oxidation of dissolved nitric oxide by dissolved oxygen occurs in aqueous solutions. Nevertheless, given the rate constants and low concentrations of dissolved NO and O₂ this reaction is considerably less rapid than in the gaseous state, where the concentration of oxygen is very high. Delivering NO in an aqueous composition is therefore highly innovative and counter-intuitive.

Nitric oxide can be produced by chemical reduction of nitrous acid. Many different reducing agents can be used to reduce nitrous acid, physiologically acceptable examples of such reducing agents include iodide anion, ascorbic acid, butylated quinone, tocopherol etc. Nitrous acid is a weak acid with pKₐ 3.4. This means that in aqueous solution at pH 3.4 nitrous acid exists as an equimolar mixture of nitrous acid (HNO₂) and nitrite (NO₂⁻).) At higher pH the equilibrium shifts in favour of nitrite anion; at lower pH the equilibrium shifts in favour of nitrous acid. Since only nitrous acid can be chemically reduced to nitric oxide the efficiency of converting nitrite into nitric oxide increases with decreasing pH. So, whilst at pH 6 the rate of such conversion is negligible, it proceeds slowly at pH 5 and is very rapid at pH <4 and especially at pH <3.

Therefore, typically the nitric oxide is generated in situ by a nitric oxide generating system, preferably by providing a nitrite in an acidic environment.

In a preferred embodiment the nitric oxide generating system comprises a reducing agent in an acidic environment together with a nitrite.

It will be appreciated that nitrite has a pKa of 3.4 (at 25° C.). Thus, nitrite can act as a buffer, capable of maintaining pH in the range between 3 to 4. Thus, there is no particular need for an additional buffer and preferably the nitrite in the aqueous composition is the only component which has a pKa of from 1 to 4. Therefore, preferably the dressing is free of any additional materials having a pKa of from 1 to 4.

A special category of reducing agents that react with nitrite in acidic environment are thiols. Reaction between thiols and nitrite in acidic environment does not result in nitrous acid reduction and immediate generation of nitric oxide, as in the case of other reducing agents. Instead, thiols are nitrosylated by the nitrosonium cation (NO⁺) which is another species generated from nitrite in acidic conditions to produce an S-nitrosothiol. Preferred thiols are thioglycerol (especially monothioglycerol, dithioglycerols and trithioglycerols), cysteine and thioglucose. Monothioglycerol (especially alpha-monothioglycerol) is most preferred.

S-Nitrosothiols (sometimes referred to simply as nitrosothiols) are compounds capable of releasing nitric oxide. S-nitrosothiols can be produced by nitrosating thiols using either N₂O₃ (equation 2) or nitrosonium cation (equation 3) as the nitrosating agent:

R—SH+N₂O₃→R—SNO+NO₂³¹ +H⁺ (2)

R—SH+NO⁺→R—SNO+H⁺ (3)

Whilst the process using N₂O₃ as the nitrosating species is very significant in vivo the second process is useful for production of nitrosothiols in vitro. The nitrosonium cation can be generated from nitrite at acidic pH:

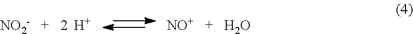

S-nitrosothiols can thus be easily produced in a laboratory by mixing a thiol (e.g.

glutathione or thioglycerol) with a source of nitrite (e.g. potassium nitrite) in acidic solution. The reaction proceeds at pH <6, the rate of the reaction increasing with the acidity of the solution:

R—SH+NO₂⁺H⁺→R—SNO+H₂O (5)

Nitrosothiols can release free nitric oxide by spontaneous decomposition:

2O═N—S—R→2NO+R—S—S—R (6)

The rate of decomposition varies considerably depending on the side chain of the thiol. For example, whilst S-nitrosocysteine can be totally decomposed within minutes under normal conditions, it takes hours/days to achieve 100% decomposition of S-nitrosoglutathione. The decomposition is generally accelerated in the presence of Copper or mercury cations. Preferably copper ions (e.g. Cu⁺ or Cu²⁺) are present.

S-nitrosothiols may be provided as they are or may be generated in situ by reacting together a nitrite and a thiol.

Suitable S-nitrosothiols include S-nitrosoglutathione (preferably S-nitroso-L-glutathione, as this is the physiologically important version), S-nitrosocysteine, S-nitroso-N-acetylcysteine, S-nitrosocaptopril, S-nitrosomercaptoethylamine, S-nitroso-3-mercaptopropanoic acid, S-nitroso-D-thioglucose and S-nitroso-N-acetyl-D, L-penicillamine.

The invention particularly relates to treatment by topical application of nitric oxide to psoriasis on the skin of a human or animal. Typically the aqueous composition comprising nitric oxide is delivered by means of a skin dressing. The term “skin dressing” covers dressings such as patches, plasters, bandages and gauze etc. for use in connection with transdermal delivery of agents. The term also includes material in amorphous or liquid form such as gels, creams, emulsions, sprays and foams. The term covers dressings for application to body surfaces generally, particularly the skin including the scalp.

The skin dressing may optionally be combined with known treatments for psoriasis, particularly known topical treatments, as desired.

Such a skin dressing may simply be applied to the region of skin exhibiting psoriasis so that nitric oxide passes from the dressing into the underlying skin. Dressings may be replaced every six to twelve hours.

The exact quantity of nitric oxide delivered to a skin site is difficult to measure but dressings which generate up to 10 mM, even up to 5 mM or even up to 2 mM nitric oxide, were found to give a significant improvement in psoriasis symptoms.

The or each dressing component may be in the form of a layer, e.g. in the form of a sheet, slab or film, that may be produced from an amorphous material, not having any fixed form or shape, that can be deformed and shaped in three dimensions, including being squeezed through a nozzle.

The aqueous composition will typically comprise more than one component. Preferably at least two components contain materials which react together when brought into contact at the skin site to be treated as part of the nitric oxide generating system. For example a first component comprising a source of acidity and a second component comprising a nitrite salt. In this embodiment the second component is preferably not acidic with a pH of from 5 to 12, preferably from 6 to 11, more preferably from 7 to 10.

The or each dressing component conveniently comprises a carrier or support, typically in the form of a polymeric matrix. The carrier may be solid or amorphous, as discussed below.

The carrier or support conveniently comprises a hydrated hydrogel. A hydrated hydrogel means one or more water-based or aqueous gels, in hydrated form. A hydrated hydrogel thus includes a source of water, for activation of the dressing.

Suitable hydrated hydrogels are disclosed in WO 03/090800. The hydrated hydrogel conveniently comprises hydrophilic polymer material. Suitable hydrophilic polymer materials include polyacrylates and methacrylates, e.g. as supplied by First Water Ltd in the form of proprietary hydrogels, including poly 2-acrylamido-2-methylpropane sulphonic acid (poly-AMPS) and/or salts thereof (e.g. as described in WO 01/96422), polysaccharides e.g. polysaccharide gums particularly xanthan gum (e.g. available under the Trade Mark Keltrol), various sugars, polycarboxylic acids (e.g. available under the Trade Mark Gantrez AN-169 BF from ISP Europe), poly(methyl vinyl ether co-maleic anhydride) (e.g. available under the Trade Mark Gantrez AN 139, having a molecular weight in the range 20,000 to 40,000), polyvinyl pyrrolidone (e.g. in the form of commercially available grades known as PVP K-30 and PVP K-90), polyethylene oxide (e.g. available under the Trade Mark Polyox WSR-301), polyvinyl alcohol (e.g. available under the Trade Mark Elvanol), cross-linked polyacrylic polymer (e.g. available under the Trade Mark Carbopol), celluloses and modified celluloses including hydroxypropyl cellulose (e.g. available under the Trade Mark Klucel EEF), sodium carboxymethyl cellulose (e.g. available under the Trade Mark Cellulose Gum 7LF) and hydroxyethyl cellulose (e.g. available under the Trade Mark Natrosol 250 LR).

Mixtures of hydrophilic polymer materials may be used in a gel.

In a hydrated hydrogel of hydrophilic polymer material, the hydrophilic polymer material is desirably present at a concentration of at least 1%, preferably at least 2%, more preferably at least 5%, yet more preferably at least 10%, or at least 20%, desirably at least 25% and even more desirably at least 30% by weight based on the total weight of the gel. Even higher amounts, up to about 40% by weight based on the total weight of the gel, may be used.

Good results have been obtained with use of a hydrated hydrogel of poly-AMPS and/or salts thereof in an amount of about 30% by weight of the total weight of the gel and with Carbopol™ polyacrylic acid amorphous gels.

The hydrated hydrogel material is typically in the form of a solid layer, sheet or film of material that is typically cross-linked, and that may incorporate a mechanical reinforcing structure. The size and shape of the layer, sheet or film can be selected to suit the intended use of the dressing. Thicknesses in the range 0.05 to 5 mm, preferably 0.5 to 3 mm are particularly suitable.

Alternatively, the hydrated hydrogel may be in the form of an amorphous gel not having a fixed form or shape, that can be deformed and shaped in three dimensions, including being squeezed through a nozzle. Amorphous gels are typically not cross-linked or have low levels of cross-linking. A shear-thinning amorphous gel may be used. Such a gel is liquid when subjected to shear stress (e.g. when being poured or squeezed through a nozzle) but set when static. Thus the gel may be in the form of one or more pourable or squeezable components that may be dispensed, e.g. from a respective compressible tube or a syringe-like dispenser, comprising a piston and cylinder, typically with a nozzle of about 3 mm diameter. Such a gel or gels may be applied in the form of a surface layer, and contacts the psoriasis surface.

A typical example of an amorphous gel formulation is: 15% w/w AMPS (sodium salt), 0.19% polyethylene glycol diacrylate and 0.01% hydroxycyclohexyl phenyl ketone, with the volume made up to 100% with analytical grade DI water. The reagents are thoroughly mixed and dissolved, then polymerised for between 30-60 seconds, using a UV-A lamp delivering approximately 100 mW/cm², to form the required hydrogel. This may be contained in plastic syringes from which the amorphous gel may then be dispensed from a syringe to a target site, as a surface layer.

An example of a two-component amorphous gel formulation is: a first gel comprising aqueous Carbopol 974P NF (4.5% w/w) with 26 mM calcium chloride and 100 mM monothioglycerol at pH 4.2, and a second gel comprising aqueous Carbopol 974P NF (1.5% w/w) and 100 mM potassium nitrite and 10 mM copper (II) nitrate at pH 10.0.

An example of a two-component emulsion is: (i) a first component comprising an emulsion prepared from a mixture of a first phase of Petrolatum, Galenol 1618 DSN, Drakeol (R) 35 and silicone fluid, and a second phase comprising aqueous acetate buffer (pH 4), phenoxyethanol and monothioglycerol, (ii) a second component comprising an emulsion prepared from a mixture of a first phase of Petrolatum, Galenol 1618 DSN, Drakeol (R) 35 and silicone fluid, and a second phase comprising Tris-HCl buffer (pH7.2), phenoxyethanol, nitrite salt and copper (II) nitrate.

While it is generally preferred to use a hydrated hydrogel or emulsion as the carrier or support, the carrier or support may instead comprise material in dry condition, with the nitric oxide generating system typically present in a dried polymeric matrix.

For example a nitric oxide donor composition, e.g. an S-nitrosothiol, could be provided in a dried condition, only to be activated as a nitric oxide generating system on being wetted when applied to the skin surface. A particularly suitable wetting system involves the addition of an acidic aqueous composition, optionally comprising metal ions such as Fe²⁺, Cu²⁺ and/or Zn²⁺.

Dry condition means that there is no free water in the material, such that no significant or measurable water loss occurs through evaporation under normal ambient conditions of temperature, pressure and humidity. Dry condition includes desiccated condition, which is an extra thoroughly dried condition. Desiccated condition means a condition maintained by storage in an environment enclosed by a moisture impermeable barrier, wherein the material is kept scrupulously free of water by means of an added desiccant.

Because the material is in dry condition the reagent, e.g. an S-nitrosothiol, a nitrite or thiol, is in stable condition and is retained in the material. The material can be stored under suitable conditions for an extended period of time, with the reagent remaining stable therein.

A suitable material to form part of a solid dressing component is a polymer material.

One preferred polymer material comprises polyvinyl alcohol (PVA). PVA has convenient and acceptable properties for skin treatment use, e.g. being non-toxic. PVA is also easy to handle and use, readily forming a film on drying of a PVA solution in water, with the resulting film being easy to handle. PVA is also readily available and low cost. Cross-linking is not required to form a solid material, e.g. in the form of a film, although cross-linking may optionally be employed. PVA is available in a wide range of grades based on molecular weight and degree of hydrolysis, which affect the physical properties of the material. Appropriate grades of PVA can be readily selected to produce a polymer product having desired properties for a particular intended use. For example, for use in skin dressings, good results have been obtained by use of PVA with a molecular weight in the range 100,000 to 200,000, substantially fully hydrolysed (98-99% hydrolysed), e.g. in the form of code 36,316-2 from Sigma-Aldrich, in non-cross-linked form and also with PVA with a molecular weight in the range of from 31,000 to 50,000 (87-89% hydrolysed) e.g. in the form of code 363073 from Sigma-Aldrich.

Another suitable polymer material comprises polyvinylpyrrolidone (PVP). The properties of PVP are very similar to those of PVA, and PVP is also acceptable for skin treatment use. PVP is readily available in a range of different molecular weights. Appropriate grades of PVP can be readily selected. For example, good results have been obtained using a PVP having a molecular weight average of 360,000, e.g. in the form of code PVP360 from Sigma, in a non-crosslinked form.

Such a solid dressing component is conveniently in the form of a sheet, layer or film, typically having a thickness in the range 0.01 to 1.0 mm, preferably in the range 0.05 to 0.5 mm. The solid material may optionally include a support to provide rigidity when wet.

Such solid polymer materials are conveniently made by mixing a solution of a polymer (e.g. an aqueous solution of PVA and/or PVP) and reagent, and drying the mixture to produce a solid material, e.g. forming a film by a casting procedure. Suitable techniques are well known to those skilled in the art.

Practical difficulties arise in incorporating a thiol in a poly-AMPS hydrogel, so this reagent is instead generally provided in a carrier comprising dry material as discussed above, e.g. a dried PVA polymeric matrix.

Thus, in one preferred embodiment the invention comprises a first component comprising a layer of hydrated hydrogel, preferably poly-AMPS and/or salts thereof, containing a source of nitrite, e.g. potassium nitrite, and a second component comprising a dry polymeric matrix, preferably dried PVA, containing a thiol, e.g. monothioglycerol. The first component is preferably used in contact with the skin, as the hydrated hydrogel has beneficial properties for skin contact, as discussed above, with the second component being placed on top of the first component. Provided the components are kept separate prior to use, the dressing remains in non-activated condition. However, when the two components are brought into contact, this has the effect of activating the dressing.

In a preferred embodiment, on activation of the aqueous composition, e.g. in the form of a dressing, nitrite starts diffusing from the first component (or primary layer) into the second component (or secondary layer), and thiol diffuses in the opposite direction. Mixing of the nitrite with the thiol in acidic solution results in generation of S-nitrosothiol. If the thiol is L-glutathione, then the product of reaction is S-nitroso-L-glutathione. Once produced, the S-nitrosothiol is released from the dressing into the surrounding environment, e.g. onto the surface of the psoriatic lesion, where it decomposes to produce nitric oxide, with consequential beneficial effects.

In another preferred embodiment, the materials are provided in two separate amorphous gels which may be intimately mixed together at the point of application on the skin wherein the above reaction takes place.

The invention will now be illustrated in the following examples.

### EXAMPLES

### Overall Study Design

This was a non-blinded, internally controlled study, with 4 subjects.

Inclusion criteria: Age 16 to 65 years. Subjects were required to have at least two discrete areas of plaque psoriasis (one for test gel and one for control gel).
Exclusion criteria: Subjects receiving systemic nitrate medication and/or Sildenafil™ or suffering from cardiovascular disease.

### Treatments and Drug Handling

The test material was a two component, water-based gel, which was mixed on the skin at the time of use to generate nitric oxide.

The constituents of the two gels of the TEST material were:
a. Aqueous carbopol 974P NF polymer (4.5% w/w) with 26 mM calcium chloride and 100 mM alpha-mono thioglycerol, at pH 4.2.
b. Aqueous carbopol 974P NF polymer (1.5% w/w) with 100 mM potassium nitrite and 10 mM copper nitrate, at pH 10.

The constituents of the two gels of the CONTROL material were:
a. Aqueous carbopol 974P NF polymer (4.5% w/w) with 26 mM calcium chloride, at pH 4.2.
b. Aqueous carbopol 974P NF polymer (1.5% w/w) with 20 mM calcium chloride, at pH 10.

The two gels were supplied in a dual chamber, pump dispenser with mixing head, set to dispense a 50:50 mix of the two gels. The dispenser was set to deliver approximately 0.3 ml of each gel per pump depression, i.e. 0.6 ml in total.

Two sites with plaque psoriasis on each patient were chosen. Patients were their own controls and their two sites were randomised to the TEST and CONTROL application by selection from a sealed envelope. One site received the TEST gel and the other the CONTROL gel (carbopol polymer vehicle). There were equal numbers of left/right side TEST treatments. Subjects did not know which site was receiving TEST gel and which CONTROL gel.

Before treatment each site was measured, traced and photographed and its overall condition assessed. Subjects were given TEST gel and CONTROL gel. The sites for application of TEST gel and CONTROL gel were chosen at random. The two treatments areas were marked. Treatment was applied twice per day for 6 weeks. Subjects were seen at 1 and 3 weeks after treatment commenced and at completion at 6 weeks. At each visit, the TEST and CONTROL application sites were assessed (measured, traced and photographed).

### Psoriasis Symptom Severity—Scoring System:

The status of the psoriatic plaques was assessed and scored using the following system: “Psoriasis symptom severity”=[L×W]×[E+T+S]
where [L×W] represents the overall area of the affected plaque in cm2 (as length×width), and
E represents clinician assessment of erythema (using 0-5 scale),
T represents clinician assessment of plaque thickness (0-5scale) and
S represents clinician assessment of plaque scaling (0-5 scale)

### Results

Over the six weeks of the study:

Patient 1's psoriasis symptom severity score for the plaque treated with TEST gel reduced from 100% to 49%, whereas the area treated with CONTROL reduced to 96% of its original value.

Patient 2's psoriasis symptom severity score for the plaque treated with TEST gel reduced from 100% to 21%, whereas the area treated with CONTROL reduced to 81% of its original value.

Patient 3's psoriasis symptom severity score for the plaque treated with TEST gel reduced from 100% to 22%, whereas the area treated with CONTROL reduced to 89% of its original value.

Patient 4's psoriasis symptom severity score for the plaque treated with TEST gel reduced from 100% to 48%, whereas the area treated with CONTROL increased to 106% of its original value.

Thus, all 4 patients experienced a significant reduction in the severity of their symptoms for plaques which received the TEST gel application (change from 100% on entry to study, to 49%, 21%, 22% and 48%, giving an average reduction to 35% of original severity). Hence, the severity of their symptoms reduced by 65% on average with the TEST gel over six weeks.

The plaques which received the CONTROL gel manifested much less change in the severity of their symptoms (changing from 100% on entry to study, to 96%, 81%, 89% and 106% on exit, i.e. an average reduction to 93% of the original level of severity). Hence, the severity of their psoriasis symptoms reduced by 7% on average with the CONTROL gel over six weeks.

1-16. (canceled)

17. A composition for treating psoriasis, comprising:
an aqueous composition configured to deliver nitric oxide to a patient's skin.

18. A skin dressing for treating psoriasis, comprising:
a nitric oxide generation system configured selectively to generate in situ nitric oxide in an aqueous composition for delivery to a patient's skin.

19. The composition of claim 18, wherein the nitric oxide generation system comprises a nitrite in an acidic environment.

20. The skin dressing of claim 18, wherein the nitric oxide generation system comprises two components that when brought selectively together initiate a reaction for generation of nitric oxide.

21. The skin dressing of claim 20, wherein the two components comprise a first component comprising a source of acidity and a second component comprising a nitrite salt.

22. The skin dressing of claim 20, wherein the two components comprise a first component comprising a nitrite and a second component comprising a reducing agent.

23. The skin dressing of claim 22, wherein the reducing agent comprises a thiol that reacts with nitrite to generate an S-nitrosothiol.

24. The skin dressing of claim 23, wherein the thiol comprises monothioglycerol.

25. The skin dressing of claim 22, wherein the nitrite is the only component in the nitric oxide generation system which has a pKa of from 1 to 4.

26. The skin dressing of claim 18, wherein the nitric oxide generation system comprises a nitrite and a reducing agent.

27. The skin dressing of claim 18, wherein nitric oxide generation system comprises at least one hydrogel.

28. The skin dressing of claim 27, wherein the at least one hydrogel comprises a nitrite.

29. The skin dressing of claim 18, wherein nitric oxide generation system is configured to deliver nitric oxide in a concentration of up to 10 mM.

30. The skin dressing of claim 18, wherein the nitric oxide generation system is configured to deliver nitric oxide in a concentration of up to 5 mM.

31. The skin dressing of claim 18, further comprising at least one of a patch, a plaster, a bandage, and a gauze for transdermal delivery of the nitric oxide to a patient's skin.

32. The skin dressing of claim 18, further comprising at least one a gel, a cream, an emulsion, a spray, and a foam for direct application to a patient's skin.

33. A method of treating psoriasis, comprising:
providing the skin dressing of claim 18; and
applying the skin dressing to a psoriasis lesion on a patient's skin.

34. The method of claim 33, wherein the nitric oxide generation system comprises a first component comprising a nitrite and a second component comprising a reducing agent that when brought together react to generate nitric oxide, the method further comprising:
causing the first component and the second component to come into contact with each other to generate nitric oxide.

35. The method of claim 33, wherein the applying comprises applying the skin dressing to a plaque psoriasis lesion on a patient's skin.

36. A skin dressing, comprising:
a nitric oxide generation system configured to selectively generate in situ nitric oxide in an aqueous composition for delivery to a patient's skin, wherein the nitric oxide generation system comprises a first component comprising a nitrite salt and a second component comprising monothioglycerol that when brought together react to generate nitric oxide, wherein the nitric oxide generation system is further configured to transdermally deliver the generated nitric oxide to a patient's skin.
